(19) United States
(12) Patent Application Publication
(10) Pub. No.: US 2014/0378551 A1
(43) Pub. Date: Dec. 25, 2014

(54) Composition for the Prophylactic Treatment of Neuropathic Pain

(71) Applicant: Universite d'Auvergne, Clermont-Ferrand (FR)

(72) Inventor: Gisele Pickering, Clermont-Ferrand (FR)

(73) Assignee: Universite d'Auvergne, Clermont-Ferrand (FR)

(21) Appl. No.: 14/350,594

(22) PCT Filed: Oct. 10, 2012
(86) PCT No.: PCT/EP2012/070043

§ 371 Date: Apr. 9, 2014

(30) Foreign Application Priority Data
Oct. 11, 2011 (FR) 1159147

(51) Int. Cl. A61K 31/13

(52) U.S. Cl. CPC A61K 31/13
USPC 514/662; 564/459

(57) ABSTRACT

The invention relates to an analgesic composition containing memantine. According to the invention, such a composition is administered daily to a human patient having to undergo a surgical operation, over a period ranging from one day to a plurality of days before said surgical operation, in order to prevent and treat the development of post-operative neuropathic pain in this same patient.

### 1. FIELD OF THE INVENTION

The field of the invention is that of pharmaceutical compositions that can be used to treat pain.

More specifically, the invention pertains to a composition comprising memantine for the prevention of post-surgical neuropathic pain.

### 2. PRIOR ART

Neuropathic pain is a neurogenic pain. The symptoms are many: sensations of stinging, electric shocks, burning or painful cold in the areas of the affected nerves, hyperalgesia, allodynia, etc. Neuropathic pain is a direct consequence of a lesion or pathology affecting the sensory system: diabetes-induced neuropathy, trauma, a surgical operation, HIV or post-herpes infection, pain in a ghost limb following an amputation, etc.

Several forms of treatment have been proposed to treat neuropathic pain. However they have not always shown high efficacy, depending on the patients and the etiology of the pain.

Numerous studies have demonstrated the implication of the N-methyl-D-aspartate receptor (NMDA receptor). The NMDA receptor is a glutamate-activated ionotropic receptor. It takes the form of an open channel, permeable to calcium ions and to monovalent cations such as sodium and potassium ions. In the physiological condition of resting, the opening of the channel is blocked by a magnesium ion. The influx of calcium or sodium ions following an excitation of the nerves shifts the magnesium ion and enables the potassium ions to go out. At the structural plane, this channel is formed by four sub-units: two NR1 sub-units and two NR2 sub-units (NR2A, NR2B, NR2C or NR2D). The NR1 sub-units are constituent and always present. The NR2 sub-units for their part specify the properties of the channel such as glutamate sensitivity and permeability to calcium ions.

The NMDA receptor is ubiquitous and is implicated in numerous functions: learning and memory, synaptic plasticity, pain mechanisms, etc. Great hopes have been put on the use of antagonists to inhibit the activation of this receptor and thus treat neuropathic pain. Among the antagonists used, we can cite especially ketamine, dextromethorphan or MK-801. Although efficacious these molecules, which are antagonistic to the NMDA receptor, show toxicity that is manifested especially in psychodysleptic disorders or hallucinations.

Memantine (1-amino-3,5-dimethyl-adamantane) is a derivative of amantadine. This molecule was first used at the end of the 70s as a treatment for Parkinson's Disease, and then as a treatment for Alzheimer's Disease. Certain studies showed its positive effect in the development of neuropathic pain in rats, including neuropathic pain was induced by ligature of the sciatic nerve or by injection of formalin (Eisenberg et al, Neurosciences Letters 187, 1995; Carlton and al., Neuroscience Letters, 198, 1995). In these studies, memantine was administered during or after the operation. A beneficial effect was also reported among patients who had undergone amputation, provided that the memantine was administered just after the operation (Buvanendran et al, International Anesthesia Research Society, 107 (4), 2008).

However, other studies have dampened the enthusiasm over this observation. In an article published in 2000, a Danish team showed that memantine is inefficacious in providing relief to patients who have had a limb amputated or show nerve lesions following surgery (Nikolajsen et al., International Anesthesia Research Society, 91, 2000).

Finally, a recent review of clinical studies on humans using many NMDA receptor antagonists has shown that the molecules, the doses used and the results vary considerably. The authors of this review have concluded from this that no real conclusions can be made on the efficacy of antagonists of the NM DA receptor as regards neuropathic pain (Collins et al, Pain Medicine, 11, 2010).

Since this type of pain affects a large number of patients and causes many complications, both physical and psychological, for the patients, new ways need to be explored to prevent the appearance of neuropathic pain or at least to treat it.

Goals of the Invention

The invention is aimed especially at overcoming these drawbacks of the prior art.

More specifically, it is a goal of the invention, in at least one embodiment, to provide a composition for preventing, or at least limiting, the appearance of neuropathic pain.

It is another goal of the invention, in at least one embodiment, to provide a composition to treat neuropathic pain.

It is yet another goal of the invention, in at least one embodiment, to propose a dosage for preventing, or at least limiting, the appearance of neuropathic pain.

### 3. SUMMARY OF THE INVENTION

These goals as well as others that shall appear here below are achieved by means of an analgesic composition.

According to the invention, such a composition inhibits the phosphorylation of the tyrosine residue 1472 in the NR2B sub-unit of the NMDA receptor, said composition being intended to prevent the development of post-operation neuropathic pain in a human patient who has to undergo an operation.

It has been discovered surprisingly that the phosphorylation of tyrosine residues at positions 1472 and 1336 of the NR2B sub-unit of the NM DA receptor is a phenomenon clearly correlated with the development of neuropathic pain. More particularly, the phosphorylation of the tyrosine residue 1472 is concomitant with the phenomenon of hyperalgesia. Hyperalgesia is characterized by the sensation of heightened pain under a stimulus which is itself painful. It is therefore a common consequence of neuropathic pain, especially diabetes-induced neuropathies. Although the molecular mechanisms of neuropathic pain in diabetes are not yet clearly identified, it has been discovered that the inhibition of the protein kinases, Src and FAK, responsible for the phosphorylation of the tyrosine residues 1472 and 1336 of the NR2B sub-unit, result in a diminishing of the phenomenon of hyperalgesia.

According to the invention, such a composition comprises memantine.

Memantine significantly inhibits the phosphorylation of the tyrosine residue at position 1472 (denoted as pTyr¹⁴⁷²) of the NR2B sub-unit of the NMDA receptor. It is relatively better tolerated by patients. Chronic treatment administered intrathecally with an inhibitor of the protein kinase Src, PP2 (formula: 4-amino-5-(4-chlorophenyl)-7-(dimethylethyl) pyrazolo [3,4-d] pyrimidine) or an anti-sense oligodeoxynucleotide directed against the protein FAK abolishes the mechanical hyperalgesia induced by diabetes. This treatment furthermore reduces phosphorylation of the Tyr¹⁴⁷² residue, suggesting a major role for Src and FAK in this context of pain.

In one embodiment, such a composition comprises 4 mg and 17 mg of pure memantine.

In another advantageous embodiment, the composition according to the invention comprises 5 mg and 20 mg of memantine hydrochloride.

According to the invention, such a composition is present in a form of a capsule or an oral or drinkable solution. These galenic forms indeed have the advantage of being easy to administer. The composition can be taken by the patient alone at home and does not require the intervention of medical staff.

In another advantageous embodiment, the composition can take an injectable form and can be administered to the patient continuously or in the form of a bolus, through perfusion of liquid memantine or liquid memantine hydrochloride.

An object of the invention is also an analgesic composition containing memantine. According to the invention, such a composition is administered daily, to a human patient who is to undergo a surgical operation, for a period ranging from one to several days before the surgical operation in order to prevent and treat the development of post-operative neuropathic pain in this patient.

Although the literature referred on the subject cites only the use of memantine in per-operative or post-operation conditions, i.e. during or after the operation, the inventors have discovered that, surprisingly, the daily administration of memantine, for a period ranging from one to several days before the operation, efficaciously limits the appearance of neuropathic pain. Studies among rats have shown that the administration of memantine during or after the operation limits the appearance of symptoms of neuropathic pain but only dose-dependently and only for a determined period of time at the end of which the symptoms reappear (Eisenberg et al, Neurosciences Letters 187, 1995). Although this document uses the terms “prophylaxis” and “prevention”, the administration referred to is administration during the operation, i.e. per-operative administration of memantine and not administration before the surgical operation as understood in the invention.

On the contrary, the inventors have shown that the prophylactic administration of memantine, i.e. for one or more days before the operation, and not solely during or after said operation, prevents the appearance of symptoms related to neuropathic pain, namely hyperalgesia, allodynia, mechanical disorders, as well as the cognitive disorders usually encountered.

The term “prophylaxis” in the literature is often used to designate per-operative administration, i.e. administration during an operation at the time of anesthesia associated with the operation, or just after the performance of this surgical operation. As understood in the invention and in the following description, the term “prophylaxy” is used to designate the administration to the patient of the composition according to the invention for one and more days before the surgical operation. In other words, as understood in the invention, a preventive or prophylactic administration consists of the administration of the composition according to the invention to the patient to be operated on, before his or her operation. The patient therefore receives a daily dose of the composition according to the invention on the eve of his or her operation or even during the days that precede it.

Hitherto, the clinical practice has been to administer memantine either at the time of putting the patient under anesthesia or during the surgical operation itself.

This distinction is important since it is because of this administration done during one or more days before the operation that the desensitizing of the NMDA receptor is efficacious and that the patient develops no symptoms or hardly any symptoms related to neuropathic pain.

The patient therefore gains in quality of life and his or her consumption of antalgics is thereby considerably reduced. This also enables the patient to recover more rapidly from surgery since his mechanical and sensory pain is diminished. The harmful consequences of such chronic pain, at the bearable pain threshold, on the patient's physical and mental health are thereby considerably limited.

The term “pre-operative” administration is understood to mean administration carried out on eve of the operation or several days before the operation.

The term “per-operative administration” is understood to mean administration carried out during the operation or at the time of the anesthesia related to the operation.

In one advantageous embodiment, the composition according to the invention is administered for a period of 1 day to 42 days before said surgical operation.

Preferably, the composition according to the invention is administered for a period ranging from 1 day to 28 days before said surgical operation.

The analgesic composition fulfils it prophylactic role against pain when it is administered four weeks before the operation.

In an even more preferred way, the composition according to the invention is administered for a period of 7 days to 14 days before said surgical operation.

Administering the composition for a period of one to two weeks before the operation avoids the sensitizing of the patient's NMDA receptors and thus prevents or least limits the development of neuropathic pain. At the same time it also limits the consumption of medicine over time, a factor that is always positive for the patient.

In one advantageous embodiment, the pre-operative administration of a composition according to the invention is done in successive stages of increasing doses.

Since the NMDA receptor is implicated in many mechanisms of the central nervous system, the patient's organism needs to be habituated gradually. This method of titrating the composition according to the invention is aimed at determining the maximum dose that has satisfactory efficacy for the patient while at the same time preventing undesirable effects related to any medicine acting on the central nervous system. As understood in the invention, the composition is administered in one-week stages, in increasing the dose by 5 mg/day at each stage.

Preferably, the composition of the invention is administered in the form of memantine hydrochloride in a dose ranging from 5 mg/day to 20 mg/day.

More specifically, the composition according to the invention is administered for a duration of 28 days preceding the operation, in a dose of:
5 mg/day of memantine hydrochloride in the first week,
10 mg/day of memantine hydrochloride in the second week,
15 mg/day of memantine hydrochloride in the third week,
20 mg/day of memantine hydrochloride in the fourth week.

As understood in the invention, the dose of memantine hydrochloride to be administered to the patient can go up to 30 mg/day. In this case, the composition according to the invention will be administered for a period of 42 days before the operation on the patient in order to comply with the stages of gradual increase by 5 mg per week.

It can also be envisaged, as understood in the invention, to prescribe higher doses going up to 55 mg/day of memantine to a patient, provided that the gradual increase of 5 mg per week is complied with.

To be specific, a dose of:
5 mg of memantine hydrochloride is equivalent to a dose of 4.15 mg of pure memantine,
10 mg of memantine hydrochloride is equivalent to a dose of 8.31 mg of pure memantine,
15 mg of memantine hydrochloride is equivalent to a dose of 12.46 mg of pure memantine,
20 mg of memantine hydrochloride is equivalent to a dose of 16.62 mg of pure memantine,
25 mg of memantine hydrochloride is equivalent to a dose of 20.77 mg of pure memantine,
30 mg of memantine hydrochloride is equivalent to a dose of 24.93 mg of pure memantine.

These doses are the maximum doses tested clinically and are well supported and tolerated by the patients. They show efficacy of action without impairing the patient's mechanical and cognitive capacities (Sang et al., 2002).

In another embodiment of the invention, the composition according to the invention is administered in the form of pure memantine in a dose ranging from 4.15 mg/jour to 24.93 mg/ day.

In a more preferred embodiment, the composition according to the invention is furthermore administered post-operatively.

Advantageously, the composition according to the invention is also administered during the operation in a dose corresponding to the stage reached during a pre-operative stage.

Administering the composition according to the invention to the patient after the operation prolongs its beneficial effect by maintaining the NMDA receptors in a deactivated state following the trauma of the operation.

Preferably, the post-operative administration of the composition according to the invention is done at a constant dose.

Indeed, for the preventive effect of the composition to be fully achieved, the titration plateau should be reached before the operation. The administration of memantine is then done at a constant dose, said dose corresponding to the plateau reached.

Advantageously, the composition of the invention is then administered daily and post-operatively at a constant dose ranging from 15 mg/day to 30 mg/day.

Preferably, the composition according to the invention is also administered post-operatively for a duration of one to 16 days.

According to the invention, the post-operative administration of the composition according to the invention for a maximum duration of 16 days is enough to maintain the beneficial effects of the prophylaxis and to limit the appearance of neuropathic pain and associated symptoms (hyperalgesia, allodynia, mechanical disorders, etc). However, it is understood that the post-operative administration of memantine can be extended beyond 16 days if the patient suffers persistent pain and can be continued until the complete disappearance of the residual pain.

In one advantageous embodiment, such a prophylactic composition is administered orally, in the form of a capsule or a drinkable solution.

This mode of administration simplifies patient's task in following up on his treatment at home. The capsules can furthermore be slow-release capsules.

Preferably, the composition according to the invention is characterized in that it is administered to the patient in the morning.

It can be taken equally well during or outside mealtimes. However, it is important that memantine should be taken at regular times in order to keep the NMDA receptors in a deactivated state.

In another embodiment of the invention, the composition according to the invention is administered by perfusion in a concentration ranging from 5 mg/day/kg to 20 mg/day/kg.

According to the invention, the prophylactic composition is intended for administration to a patient who has to undergo surgery that can cause neuropathic pain, said surgery being one of the following: ablation of a limb, of a part of a limb, an organ or a part of an organ.

The amputation of a limb or ablation of a part of an organ is systematically accompanied by the elimination of a part of the nerves associated with this limb or this part of an organ. This results in heavy trauma for the patient's organism that takes the form of intense and chronic pain. There is also the known phenomenon of ghost or phantom limb pain, which is a phenomenon that concerns 90% to 98% of amputated patients. It is called ghost limb pain because the patient feels intense pain in what seems to him to be his amputated limb although he knows that there is nothing real about this pain. This pain comes a constant and intense stimulation of the nerves linked to a particular anatomical region, this region being associated with a limb or an organ in the brain's somato-sensory map.

Administering a composition according to the invention prophylactically, i.e. for a period of 1 to 42 days before the operation, preferably for a period of 1 to 28 days before the operation and even more preferably for a period of 7 to 14 days before the operation prevents or at least greatly limits the appearance of this neuropathic pain. The patients concerned by pains of this type are extremely numerous, having suffered amputation of a limb or a part of an organ following ischemia of the limb or an infection, amputation of a part of an organ to eliminate a tumor as in the case of cancers of the breast, bone, liver, lung, muscles, kidneys, etc.

Yet another object of the invention is a method for processing and preventing post-operative neuropathic pain, in a human patient who has to undergo a surgical operation, by the prophylactic administration of memantine.

The method according to the invention prevents the appearance of the symptoms of neuropathic pain or at least limits their intensity through the preventive and daily administration of memantine to a patient. The term “preventive administration” is understood to mean that the memantine must be administered to the patient before his operation and not only during or just after the operation. This pre-operative administration prevents the sensitizing of the NMDA receptors implicated in the development of neuropathic pain and the appearance of the associated symptoms such as hyperalgesia, allodynia and mechanical problems.

In one advantageous embodiment, the method according to the invention provides for the daily administration of memantine for a period of 1 to 28 days before the operation to which said human patient is to be subjected.

Preferably, the method of the invention provides for the pre-operative administration of memantine in successive stages of increasing doses.

This administration of memantine by successive stages of increasing doses, also called titration, gradually habituates the patient's organism to the inhibiting action of memantine on the NMDA receptors. Since these receptors are ubiquitous and come into play in numerous processes of the central nervous system, it is advised to have a gradual dose escalation.

According to the invention, this titration is done in one-week stages, the dose of memantine administered daily to the patient increasing by 5 mg per week.

Preferably, the method according to the invention, provides that memantine is administered in the form of memantine hydrochloride in a dose of 5 mg/day to 20 mg/day. This dose can be increased up to 30 mg/day.

In another advantageous of the embodiment of the invention, the method according to the invention provides for memantine to be administered in pure form in a dose of 4.15 mg/day to 24.93 mg/day.

The method according to the invention furthermore provides that the memantine can be administered per-operatively and post-operatively, i.e. that the memantine is administered during or after the operation.

This step enables the NMDA receptors to be kept in an inhibited state despite the trauma of the operation and the lesions inflicted on the nerve endings.

Preferably, the method according to the invention provides that the per-operative and post-operative administration of memantine is done in constant doses.

In this case, the dose used corresponds to the maximum dose at which the titration has reached. For example, if the titration has been done for 28 days, to reach a maximum dosage of 20 mg/day of memantine hydrochloride, the memantine dose used per-operatively and/or post-operatively will be 20 mg/day.

In an advantageous embodiment, the step of post-operative administration of the memantine is done for a duration of one to 16 days after the operation.

In another embodiment, the method of the invention provides that the memantine is administered in the morning.

In a preferred embodiment, the method of prevention and treatment of neuropathic pain according to the invention is applied to patients who have to undergo the ablation of a limb, a part of a limb, an organ or a part of an organ.

### 4. LIST OF FIGURES

Other features and advantages of the invention shall appear more clearly from the following description and the appended drawings, of which:

FIG. 1 is a graph representing the effects of post-operative administration of memantine on mechanical hyperalgesia among rats.

FIG. 2 illustrates the effects, by comparison with FIG. 1, of administration of memantine in pre-operative or post-operative situations on mechanical hyperalgesia among rats.

FIG. 3 is a graph representing the effects of post-operative administration of memantine on mechanical allodynia among rats.

FIG. 4 illustrates the effects, by comparison with FIG. 3, of the administration of memantine pre-operatively and post-operatively on mechanical allodynia among rats, in the form of a graph.

FIG. 5 is a graph presenting the effects of the prophylactic administration of memantine on the spatial memory of rats.

FIG. 6 is a photograph of a Western Blot gel presenting the effects of memantine on the phosphorylation of tyrosine residue 1472 of the NR2B sub-unit of the NMDA receptors among rats.

FIG. 7 is a graph presenting the results of a phase 2 clinical trial among 15 patients who received either a placebo composition or a composition according to the invention.

### 5. DESCRIPTION OF ONE EMBODIMENT OF THE INVENTION

The general principle of the invention relies on the preventive administration, i.e. the administration for one or more days before a patient's scheduled operation, of an NMDA receptor inhibitor. More particularly, this inhibitor can be an inhibitor of phosphorylation of tyrosine 1336 or 1472 residue of the NR2B sub-unit of the NMDA receptor. This inhibitor can also be an antagonist of the NMDA receptor such as memantine. This pre-operative administration, i.e. administration of memantine between one or more days before the operation, averts the sensitizing of the NMDA receptors implicated in the development of neuropathic pain. This absence of early sensitization not only limits the intensity of the symptoms associated with neuropathic pain but also prevents these symptoms from arising.

6.1 Study of the effects of memantine on neuropathic pain induced by spinal nerve ligation among rats

The effects of the prolonged and preventive administration of memantine were first of all studied among rats. The model used was that of the ligation of the spinal nerve L5 (herein after called SNL for spinal nerve ligation) among rats. The emergence of neuropathic pain is assessed according to the following criteria:
the behavior of the rat following the spinal nerve ligation (SNL),
cognition, and
molecular events, including the expression of phosphorylated tyrosine 1472 of the NR2B sub-unit of the NMDA receptor at the spinal level.

Spinal nerve ligation among rats causes painful neuropathy accompanied by hyperalgesia and allodynia, symptoms frequently described among patients suffering from neuropathic pain.

Hyperalgesia or hyperalgia corresponds to a sensation of increased pain felt as the result of a painful stimulus. Allodynia corresponds to a sense of increased pain felt following a stimulus that is normally painless among healthy individuals.

The study was based on the comparison of the two main groups of animals. The first group was a control group. The rats of the control group, designated as “Sham” in the graphs, underwent no operation. They are used to assess the role of the injected substances on the individual, without there being any emergence of neuropathic pain.

Two sub-groups were set up in this group:
one sub-group treated with the vehicle which is a saline solution of NaCl 0.9% by mass, in a dose of 1 mL/kg/rat/day, by intraperitoneal injection; and
one sub-group treated with memantine in a dose of 20 mg/kg/rat/day, by intraperitoneal injection.

The second group of animals comprises rats having to undergo to spinal nerve ligation and therefore likely to develop neuropathic pain. This group is referred to in the graphs by the term SNL. As in the control group, this group is divided into two sub-groups:
one sub-group treated with the vehicle, which is a saline solution of NaCl 0.9% by mass, in a dose of 1 mL/kg/rat/day, by intraperitoneal injection; and
one sub-group treated by memantine in a dose of 20 mg/kg/rat/day, by intraperitoneal injection.

Finally, among these groups and sub-groups, certain rats receive their daily injection of saline solution or memantine for four days before the operation and for three days after the operation, and other rats receive their daily injection only for three days following the operation. The number of rats in each group and in each condition is indicated in brackets in the figures.

6.1.1 Spinal Nerve Ligation (SNL)

In brief, spinal nerve ligation among rats is done in the following steps.

1. The rats receive an intraperitoneal injection of pentobarbital (6%, 1 ml/kg). Rats not anesthetized at the end of this injection will be re-anesthetized on the next day in a dose of 1.2 ml/kg. The lumbar part of the back of the rats is shaven and then disinfected (with Betadine and alcohol at 70° C.).

2. The skin is incised over about 2 cm on either side of L5, about 0.5 cm to the left of the vertebral column. The left paraspinal muscle is dissected so as to open out the lateral part of the vertebral column. The L5 transverse process is exposed and then withdrawn by means of a gouge, thus releasing the L5 nerve. This nerve is isolated on a glass micropipette of very small diameter.

3. Using a glass micropipette and a slipknot, a silk thread is passed beneath the nerve L5 and knotted around the nerve; the two ends of the thread are cut at about 3 mm from the knot.

4. Once the ligation of the nerves has been done, the muscle and the skin are re-stitched. The wound is disinfected with Betadine. An antibiotic solution (Négérol®, Céva Santé Animale, 150 ml/142 g) is sprayed over the stitched wound. A solution of NaCl 0.9% is injected subcutaneously (s.c., 10 ml/kg) to rehydrate the rat.

5. The rats are isolated in a cage under a lamp until they wake up. Pellets of food are placed in the sawdust. Then, a non-steroid anti-inflammatory treatment known as Meloxicam (Mobic®, 2 mg/kg) is administered subcutaneously for two days.

6. The animals are observed daily for any unusual behavior. The anomalies of painful sensation appear between three to 10 days after the surgery and persist for at least 28 days.

7. Seven to 14 days after the surgery, the neuropathic, hyperalgesic or allodynic rats are selected according to their response to nociceptive and non-nociceptive stimulation assessed through appropriate tests (see paragraph 6.1.2). Only animals whose nociception thresholds are reduced by at least 15% relative to the thresholds measured before surgery or those for which the application of a non-painful stimulation prompts a painful response are considered to be respectively hyperalgesic or allodynic and included in the study. The non-hyperalgesic or non-allodynic rats (about 1% to 5% of the rats operated) are discarded from the study.

6.1.2 Behavioral and Cognitive Tests Used to Select Rats Having Neuropathic Pain

### Paw Pressure Tests: Mechanical Hyperalgesia

The rats are subjected to the paw pressure tests (Randall & Selitto, 1957). The nociceptive threshold, the vocalization or the struggle response, is measured by means of a Ugo Basile (Bioseb®) analgesiometer. This analgesiometer, using a cone placed on the left rear paw of the rat, exerts increasing pressure by the movement of a weight along a graduated scale. This stimulation is stopped as soon as the animal reacts. The distance travelled by the weight in centimeters is multiplied by 30 to obtain the pain threshold in grams. The maximum pressure exerted or “cut-off” pressure is 450 g. Any diminishing of the vocalization threshold is a sign of hyperalgesia.

### Von Frey Test: Mechanical Allodynia:

Eight Von Frey filaments or hairs (Chaplan et al., 1994) are used in this study, weighing 1.4 g to 26 g. The rats are placed on the support for 15 to 20 minutes for habituation. The filaments are applied perpendicularly to the plantar surface of the rat's paw at the level of the pads, starting with the 6-gramme filament. The size of the next filaments will be determined by the response of the rat according to the Chaplan method (1994). If the rat reacts to the stimulus, the next filament will be the one situated immediately below in the range and vice versa. The experiment continues until 4 filaments have been applied after the first change in response behavior of the rat. The rats thus receive 5 to 9 stimuli in all. The response of the rats is expressed by the 50% response threshold according to the Dixon formula (1980).

### Y-Maze Test: Spatial Memory

The rat is placed in the middle of a chamber with three arms, each 40 cm long and 4 cm wide. The rat moves around freely in the different arms for five minutes and the number of entries into each of the arms is counted up. This non-invasive test is based on the exploratory behavior of the animal and on the cognitive capacity of this animal (Dellu et al., 1992).

The study takes place according to the following timeline:
D-6: Von-Frey test
D-5: paw pressure test
D-4 to D-1: pre-operative injections of memantine or NaCl (once/day) for rats of the group who have to undergo spinal nerve ligation
D0: spinal nerve ligation (SNL)
D0 to D2: post-operative injections of memantine or NaCl (once a day) for rats having undergone spinal nerve ligation as well as for rats of the control group (Sham)
D8: Y-Maze test
D8: Von-Frey test
D9: paw pressure test
D14: Von-Frey test
D15: paw pressure test
D16: Sacrifice the animals and remove spinal cord

6.1.3 Effects of Memantine at the Molecular Level:

The effects of memantine are also evaluated in the expression of the phosphorylation of tyrosine 1472 of the NR2B sub-unit of the NMDA receptor at the spinal level by the Western-blot technique.

The rats are sacrificed by decapitation. The lumbar enlargements (L4-L6) are collected and subjected to lysis at 4° C. in 400 μL of stop buffer (50 mM Hepes, pH 7.5, containing 150 mM NaCl, 10 mM EDTA, 10 mM Na₂P₂O₇, 2 mM Na₃VO₄, 100 mM NaF 1% Triton, 0.5 mM PMSF, 100 UI/mL Iniprol and 20 μM Leupeptine).

After sonication, two operations of centrifugation at 14,000 rpm at 4° C. are used to retrieve the proteins from the cytoplasm. The supernatant is retrieved and the proteins are determined by colorimetry (BC Assay UP40840A®, Interchim). They are denatured at 100° C. for five minutes in a buffer Tris 100 mM, pH 6.8, containing 6% SDS, 20% Glycerol and 20% bromophenol/(β-mercaptoethanol. Electrophoresis is used to separate the proteins on a polyacrylamide gel. The migration is done at 90 mA for two hours in the following buffer: Tris 50 mM, pH 7.4, containing 380 mM Glycine and 7 mM SDS. The proteins are then transferred to a nitrocellulose membrane (Millipore) for three hours in a buffer Tris 25 mM, pH 8.3, containing 190 mM Glycine, 20% methanol (percentage by volume). The protocols are carried out according to the recommendations of the supplier and differ according to the antibody considered.

After blocking of the non-specific sites with a solution containing 5% of non-fat dried milk, the membrane is incubated throughout the night at 4% under agitation with the antibody of interest diluted in the blocking solution, namely an anti-pTyr¹⁴⁷² NR2B antibody recognizing NR2B phosphorylated on tyrosine 1472 (dilution by volume=1/1000, Millipore).

The membrane is then hybridized for one hour under agitation with an antibody coupled with peroxydase (dilution by volume=1/10 000, Pierce) diluted in the blocking buffer.

The developing is done by chemiluminescence (Super Signal® West Pico

Chemiluminescent Substrate, Pierce). The densitometric analysis of the membranes is done by means of an image analyzer (Chemidoc™, XRS Biorad) and a computer software program (Image Lab™ Software).

The data is expressed by the ratio: intensity of the bands corresponding to the phosphorylated form of the protein of interest (in this case pTyr¹⁴⁷² NR2B)/intensity of the bands corresponding to the total form (phosphorylated and non-phosphorylated) of this very same protein. The results are expressed in percentage relative to the control, the control being the average of the values obtained on at least three spinal cords coming from animals having received the vehicle.

6.1.4 Results:

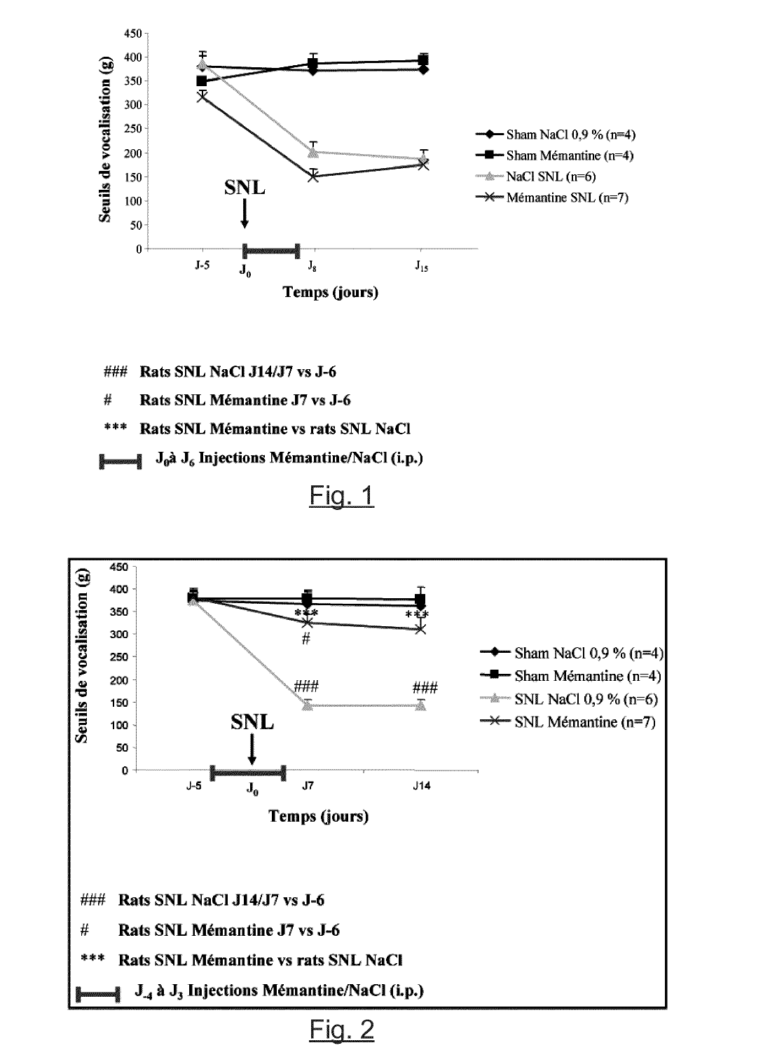

FIGS. 1 and 2 illustrates the effects of administration of memantine on mechanical hyperalgesia, assessed by means of the Randall and Sellito test. FIG. 1 presents the results obtained by rats having received post-operative injections of either memantine or the vehicle (namely saline solution of NaCl at 0.9%). FIG. 2 presents the results obtained on rats having received either the vehicle or memantine for four days before the operation and for three days after the operation. According to these two figures, memantine by itself has no influence whatsoever on the behavior of non-operated rats as compared with non-operated rats having received the vehicle. Following the ligation, the rats show obvious signs of hyperalgesia. As compared with FIG. 1, the post-operative injection of memantine is not enough to restore a normal vocalization threshold between the operated rats and the Sham rats. By contrast, rats that have received memantine preventively have a vocalization threshold very close to that of non-operated rats as can be seen in FIG. 2. The preventive injection of memantine therefore limits mechanical hyperalgesia resulting from neuropathic pain.

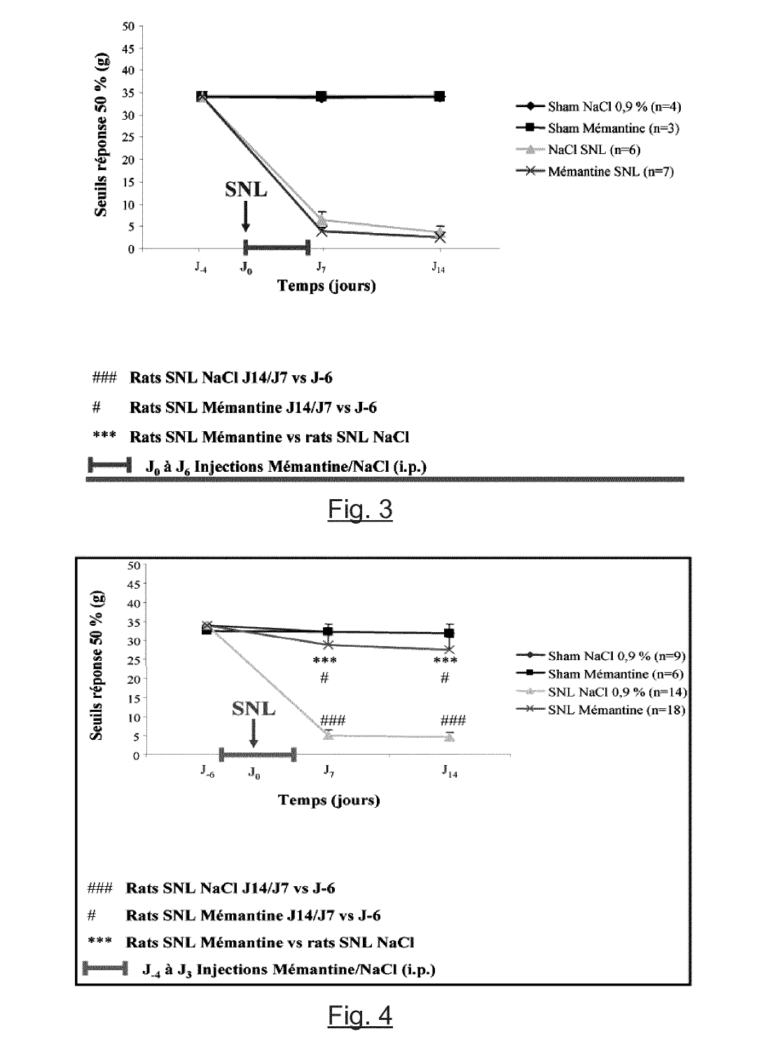

FIGS. 3 and 4 illustrate the effects of the administration of memantine on mechanical allodynia, evaluated through the Von Frey test. FIG. 3 presents the results obtained on rats having received only post-operative injections of either memantine or the vehicle (namely the saline solution of NaCl at 0.9%). FIG. 4 presents the results obtained on rats having received either the vehicle or the memantine during the 4 days preceding the operation and the 3 days following the operation. According to these two figures, it can be noted that memantine alone has no influence whatsoever on the behavior of non-operated rats as compared with non-operated rats having received the vehicle. The lines coincide in the graphs of these two figures. Following the ligation, the rats show obvious signs of allodynia. The injection of memantine solely post-operatively is not enough to restore the normal behavior of the rats as can be seen in FIG. 3. However, the prophylactic administration of memantine among operated rats restores a threshold of response almost identical to that of non-operated rats. The injection of a vehicle does not give the same result. It is therefore patently clear that prophylactic administration eliminates neuropathic pain or at least limits the development of allodynia.

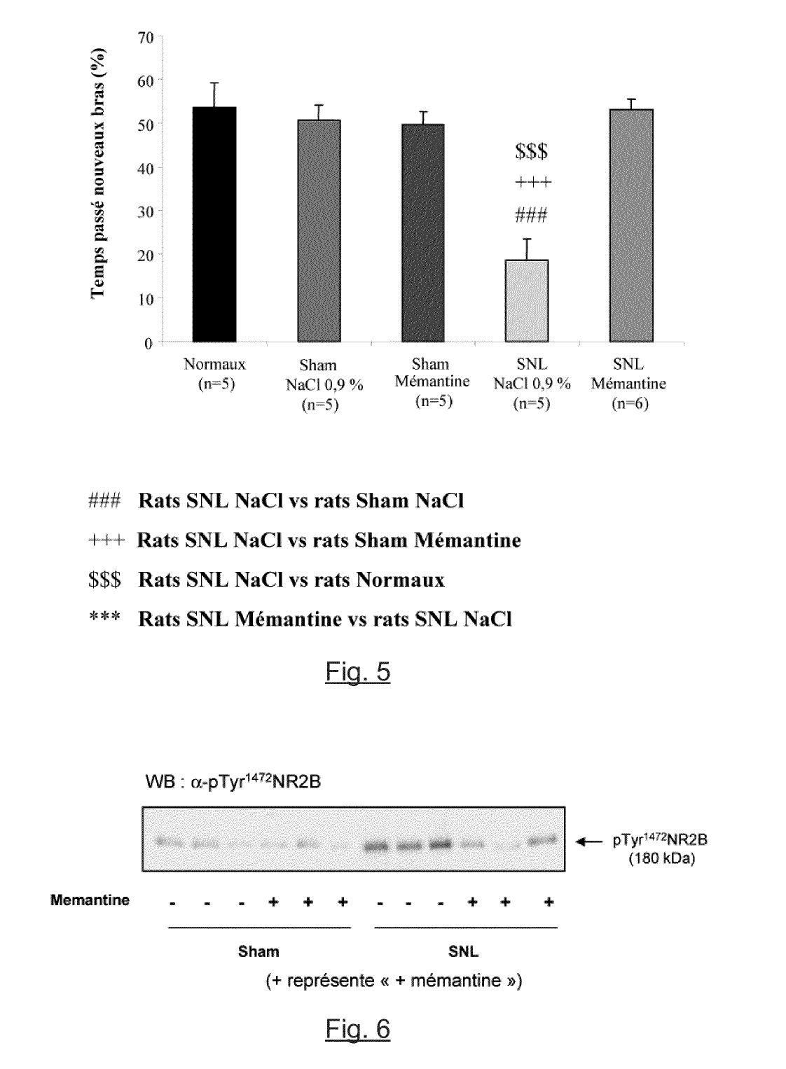

FIG. 5 represents the results of the Y-Maze test, namely the time passed in exploring the arms of the chamber (in percentage, as compared with the total time passed in the chamber). The results are presented for normal rats who have received no treatment and have undergone no operation, for rats having received an injection during the 4 days preceding their operation and the 4 days following the operation, whether memantine or the vehicle, and finally for rats having received an injection of memantine or vehicle for one week without undergoing ligation.

It is seen that the injection of the vehicle or memantine does not significantly modify the capacity of exploration of the rat as compared with normal rats. Spinal nerve ligation considerably impairs this capacity as can be noted for SNL rats that have received only the vehicle. However, this capacity is restored for rats having received memantine for the 4 days preceding their operation.

After the sacrifice of the rats, the phosphorylation of the tyrosine 1472 residue of the sub-unit NR2B is determined by Western blot technique. FIG. 6 is a photograph of a Western membrane after development of the bands. It can be noted that, in the group of the Sham rats, memantine does not significantly diminish the phosphorylation of the tyrosine 1472 residue. However, the rats treated preventively by memantine show a rate of phosphorylation of the tyrosine 1472 residue that is equivalent firstly to that of the non-treated Sham rats and is secondly far lower than that of the operated rats that have not been treated with memantine.

In conclusion, the administration of memantine before an operation prevents the appearance of neuropathic pain and its main symptoms, namely hyperalgesia, allodynia and cognitive disorders more efficaciously than does the per-operative and post-operative administration of memantine.

This result can be compared on the molecular level with an inhibition of the phosphorylation of the tyrosine 1472 residue of the NR2B sub-unit of the NMDA receptors. We can therefore conclude that the inhibition of the phosphorylation of this residue prevents the development of neuropathic pain and the associated symptoms.

6.2 Example of a protocol of administration of memantine in a patient who had to undergo an operation inducing neuropathic pain: mastectomy (tumorectomy plus axillary dissection)

A patient having undergone mastectomy for breast cancer was administered memantine in the form of memantine hydrochloride, daily and for the 28 days preceding her operation according to the following regime:
Day −28 to Day −22 (included): 5 mg/day of memantine hydrochloride,
Day −21 to Day −15 (included): 10 mg/day of memantine hydrochloride,
Day −14 to Day −8 (included): 15 mg/day of memantine hydrochloride,
Day −7 to Day 0 (included): 20 mg/day of memantine hydrochloride.

A dose of 30 mg of memantine hydrochloride was also administered to the patient on the day of her operation (Day 0) in order to maintain the blockage of the NMDA receptors by the memantine.

The dose of 20 mg/day of memantine hydrochloride was maintained for the two weeks that follow the operation in order to limit the emergence of neuropathic pain in the patient operated on.

The memantine hydrochloride was administered in the form of a capsule to be swallowed every morning during or outside meal times.

The patient's pain can be assessed according to any commonly-used method: numerical scale graduated from 1 to 10 (10 being the maximum stage of pain), Leeds sleep evaluation questionnaire (LSEQ), SF-36 quality of life questionnaire, brief pain inventory (BPI), questionnaire on neuropathic pain (DN4), Questionnaire sur la Douleur de Saint-Antoine (QDSA) or “Saint-Antoine pain questionnaire”, Hospital Anxiety and

Depression (HAD) scale, NPSI self-administered questionnaire, cognitive trail-making test A and B, DSST (Digit Symbol Substitution Test) neuropsychological test. The evaluation of the quantity of antalgics ingested by the patient after his or her operation can also be used to assess the neuropathic pain.

6.3 Administration of a composition according to the invention to patients having to undergo an operation inducing neuropathic pain: mastectomy (tumorectomy and axillary dissection) following cancer of the breast

A phase 2 clinical trial on the development and evaluation of neuropathic pain was conducted among patients having to undergo mastectomy and axillary dissection. The patients were separated randomly into two groups:
a control group who were to receive lactose as a placebo; and
a group treated by memantine hydrochloride (EBIXA®).

The clinical trial protocol is described in detail in the form referenced NTC014536314 (http://clinicaltrials.gov). Briefly, the patients received either memantine hydrochloride or the placebo according to the following protocol:
for the two weeks preceding the operation, the patients received a dose of 5 mg/day to 20 mg/day, the administered dose being increased regularly by 5 mg/day as recommended by the MA (marketing authorization) for the medicinal product EBIXA® in order to reach the dose of 20 mg/day on the day of the surgical operation;
on the day of the operation, the patients received a dose of 20 mg/day, and
for the two weeks following the operation, the patients received a constant dose of 20 mg/day.

For the two weeks preceding the operation, the patients received a dose increasing in stages of 5 mg/day of the memantine-based composition or placebo.

The patient's pain was evaluated by asking her to assess her own pain on a numerical scale ranging from 1 to 10 (10 being the maximum stage of pain) for the two weeks following the operation and for three months following the operation. This method of evaluating pain is commonly used in clinical trials. The development of neuropathic pain is also evaluated by the DN4 questionnaire on neuropathic pain, and the Questionnaire sur la Douleur de Saint-Antoine (QDSA) or “Saint-Antoine pain questionnaire”. Other methods of evaluation were also used such as the Leeds sleep evaluation questionnaire (LSEQ), the SF-36 quality of life questionnaire, the brief pain inventory (BPI), the Hospital Anxiety and Depression (HAD) scale, the NPSI self-administered questionnaire, the cognitive trail-making tests A and B, and the DSST (Digit Symbol Substitution Test) neuropsychological test.

The patients were questioned 15 days before their operation (D −15 day), at the time of their entry into clinical trials, on the day of their operation (D 0), then five days (D +5 days), 15 days (D +15 day) and three months (D +3 months) after the operation.

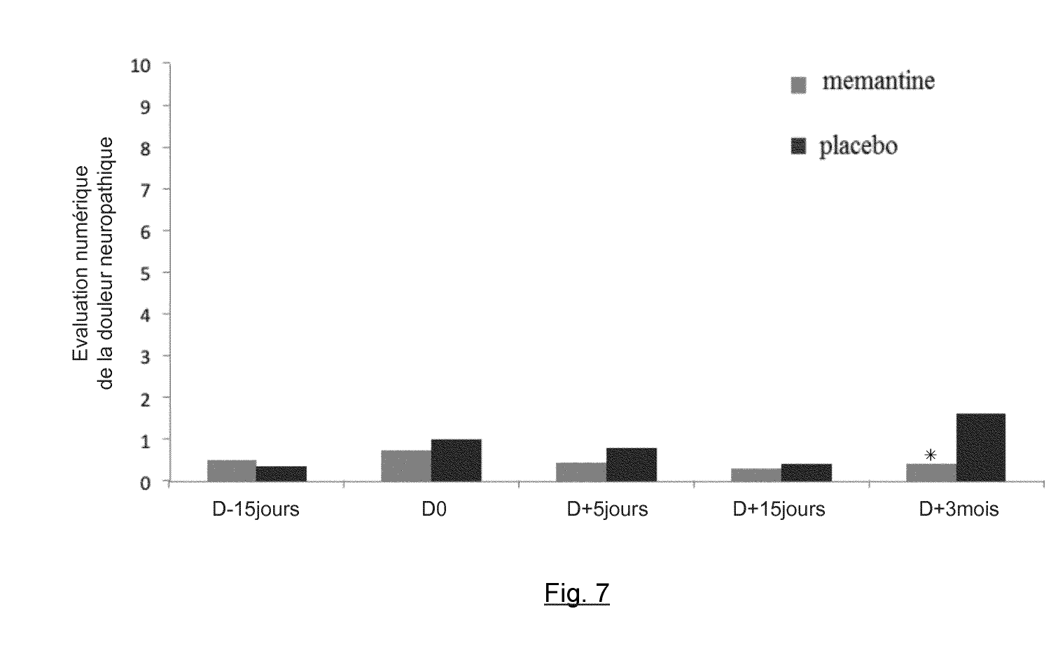

FIG. 7 is a graph showing the preliminary results obtained on the first 15 patients having taken part in these clinical trials (six patients had received the placebo composition and seven patients had received the memantine-hydrochloride-based composition), their pain being evaluated by the method of self-evaluation on a scale of 1 to 10. The day of the operation is indicated by D in the graph. The asterisk indicates a statistical significance (p<0.05).

As shown in FIG. 7, patients having received the placebo have a higher level of pain than patients having received memantine. Three months after the operation, the difference between patients treated preventively by memantine and patients treated according to the same protocol is significant although the last administration was made two weeks after the operation. Indeed, patients having received the memantine hydrochloride based composition preventively, as understood in the invention, did not show any pain (including pain between 0 and 0.5 on the scale) while the patients having received a placebo continue to show levels of pain of an average of 2 on the scale. These results are correlated by the results obtained with the same methods of evaluation.

6. Conclusion

The composition according to the invention administered preventively, i.e. one or more days before the day of the surgical operation, therefore attenuates or durably limits the development of neuropathic pain in the patient. The inventors have thus shown that the prophylactic administration of memantine, i.e. its administration before the operation and not solely at the time of the operation, greatly limits the development of neuropathic pain by the early non-sensitization of the NMDA receptors implicated in this type of pain.

The present invention also more efficaciously reduces or even prevents the suffering of patients operated. Hitherto, memantine was administered to patients at the time of their operation and this administration was continued after the operation in order to treat their pain. By administrating memantine before the operation, for a duration of one to 42 days before this operation, the inventors have found a way to limit or even prevent the development of neuropathic pain.

1. Analgesic composition containing memantine, characterized in that it is administered daily, to a human patient who is to undergo a surgical operation, for a period ranging from one to several days before the surgical operation, in order to prevent and treat the development of post-operative neuropathic pain in this patient.

2. Composition according to claim 1, characterized in that it is administered for a period of 1 day to 42 days before said surgical operation.

3. Composition according to claim 1, characterized in that it is administered for a period ranging from 1 day to 28 days before said surgical operation.

4. Composition according to claim 1, characterized in that the pre-operative administration of a composition according to the invention is done in successive stages of increasing doses.

5. Composition according to claim 1, characterized in that it is administered in memantine hydrochloride form in a dose ranging from 5 mg/day to 20 mg/day.

6. Composition according to claim 1, characterized in that it is administered post-operatively at a constant dose ranging from 15 mg/day to 30 mg/day.

7. Composition according to claim 6, characterized in that it is administered daily, post-operatively, for a duration of one to 16 days.

8. Composition according to claim 1, characterized in that it is administered orally, in the form of a capsule or a drinkable solution.

9. Composition according to claim 1, characterized in that it is administered by perfusion in a concentration ranging from 5 mg/day/kg to 20 mg/day/kg.

10. Composition according to claim 1, characterized in that it is intended for administration to a patient who has to undergo surgery that can cause neuropathic pain, said surgery being one of the following: ablation of a limb, of a part of a limb, an organ or a part of an organ.

11. Method for processing and preventing post-operative neuropathic pain, in a human patient who has to undergo a surgical operation, by the prophylactic administration of memantine comprising the step of a daily administration of memantine for a period of 1 day to 42 days before said surgical operation.

12. Method according to claim 11 providing the daily administration of memantine for a period of 1 day to 28 days before said surgical operation.

13. Method according to claim 11 providing for the pre-operative administration of memantine in successive stages of increasing doses by 5 mg/week.

14. Method according to claim 11, wherein memantine is administered in the form of memantine hydrochloride in a dose of 5 mg/day to 20 mg/day.

15. Method according to claim 11, wherein memantine can be administered per-operatively and post-operatively, in constant doses.

16. Method according to claim 11, wherein that the memantine is administered in the morning.
